(19) United States
(12) Patent Application Publication
(10) Pub. No.: US 2011/0130370 A1
(43) Pub. Date: Jun. 2, 2011

(54) Pharmaceutical composition comprising a strontium salt, vitamin D and A cyclodextrin

(75) Inventors: Gilles Briault, Saint Peravy Epreux (FR); Xavier Quenault, Baule (FR); Cécile Poirier, Orleans (FR); Jean-Manuel Pean, Orleans (FR)

(73) Assignee: LES LABORATORIES SERVIER, Suresnes Cedex (FR)

(21) Appl. No.: 12/927,733

(22) Filed: Nov. 23, 2010

(30) Foreign Application Priority Data
Nov. 27, 2009 (FR) 09/05706

(51) Int. Cl. A61K 31/59; A61P 19/08; A61P 19/02

(52) U.S. Cl. 514/167

(57) ABSTRACT

A pharmaceutical composition comprising a strontium salt, vitamin D and a cyclodextrin.

The present invention relates to a pharmaceutical composition comprising a strontium salt, vitamin D and a cyclodextrin and also to the use thereof in the treatment of bone diseases and arthrosis.

Use of strontium salts in therapy has been described, especially in patent specifications EP 0 415 850, EP 0 813 869, EP 1 534 305 and EP 1 845 082.

Compositions comprising a strontium salt and vitamin D have been described in generic manner in Patent Application WO 2004/098618.

Pharmaceutical compositions comprising strontium ranelate and vitamin D have been described in Patent Application CN 1823764.

The Applicant has found that complexing vitamin D with a cyclodextrin simultaneously improves the stability and the uniformity of content of the vitamin D within the composition.

Vitamin D is understood to be cholecalciferol (vitamin D₃), ergocalciferol (vitamin D₂), calcidiol (25-hydroxyvitamin D₃) or calcitriol (1,25-dihydroxyvitamin D₃). The vitamin D preferably used in compositions according to the invention is vitamin D₃.

Among the cyclodextrins that may be used in compositions according to the invention there may be mentioned, without implying any limitation, α-cyclodextrins, β-cyclodextrins and γ-cyclodextrins, in substituted or unsubstituted form.

Among the substituted cyclodextrins there may be more especially mentioned α-cyclodextrins, β-cyclodextrins and γ-cyclodextrins substituted by one or more methyl, hydroxypropyl or sulphobutyl ether groups

Preferred cyclodextrins are substituted β-cyclodextrins.

Among the substituted β-cyclodextrins there may be more especially mentioned HPBCDs (hydroxypropyl-β-cyclodextrins), SBECDs (sulphobutyl ether β-cyclodextrins) and methylated or partially methylated β-cyclodextrins such as DIMEB (heptakis(2,6-di-O-methyl)-β-cyclodextrin), RAMEB (randomly methylated β-cyclodextrin) or TRIMEB (heptakis(2,3,6-tri-O-methyl)-(β-cyclodextrin).

Among the strontium salts there may be more especially mentioned strontium ranelate, strontium malonate, strontium acetate, strontium L-ascorbate, strontium aspartate, strontium borate, strontium camphorate, strontium carbonate, strontium ketoglutarate, strontium citrate, strontium ethanesulphonate, strontium formate, strontium fumarate, strontium gluconate, strontium glutamate, strontium hydrogen phosphate, strontium lactate, strontium L-lactate, strontium L-malate, strontium maleate, strontium methanesulphonate, strontium nitrate, strontium oxalate, strontium phosphate, strontium propanesulphonate, strontium succinate, strontium sulphate, strontium tartrate, and also hydrates thereof.

Among the pharmaceutical compositions according to the invention there may be more especially mentioned those that are suitable for oral administration, and especially tablets and dragées to be swallowed, tablets to be chewed, effervescent tablets, dispersible tablets, sublingual tablets, capsules, and granules for sachets.

In addition to the strontium salt, vitamin D and cyclodextrin, the pharmaceutical compositions according to the invention comprise one or more excipients or carriers such as diluents, lubricants, binders, disintegrating agents, colourants, sweeteners, flavouring agents.

By way of example of excipients or carriers there may be mentioned:
as diluents: lactose, dextrose, sucrose, mannitol, sorbitol, cellulose,
as lubricants: silica, talc, stearic acid and its magnesium and calcium salts, polyethylene glycol,
as binders: aluminium and magnesium silicate, starch, gelatin, methylcellulose, sodium carboxymethylcellulose and polyvinylpyrrolidone, maltodextrin,
as disintegrants: alginic acid and its sodium salt, effervescent mixtures, carboxymethylcellulose, sodium croscarmellose,
as sweeteners: aspartame, acesulfame, sucralose.

The percentage of strontium salt in the pharmaceutical composition is preferably between 40% and 99.9% by weight inclusive.

The amount of strontium salt in the pharmaceutical composition is preferably between 200 mg and 2 g inclusive.

The amount of vitamin D₃ in the pharmaceutical composition is preferably between 5 μg (200 IU) and 175 μg (7000 IU) inclusive.

The amount of cyclodextrin in the pharmaceutical composition is preferably between 200 μg and 140 mg, more preferably between 2 mg and 70 mg, inclusive.

The ratio by weight between the amount of vitamin D and the amount of cyclodextrin is preferably between 1/40 and 1/800 inclusive.

The present invention relates also to use of the pharmaceutical compositions according to the invention in the treatment of bone diseases, more especially osteopenia and osteoporosis, and in the treatment of arthrosis.

### ABBREVIATIONS/ACRONYMS

DIMEB heptakis(2,6-di-O-methyl)-β-cyclodextrin. The degree of substitution of DIMEB is 14 methyl groups/cyclodextrin.
HPBCD hydroxypropyl-13-cyclodextrin.
RH relative humidity
RAMEB randomly methylated β-cyclodextrin (RAndomly MEthylated Beta-cyclodextrin). The average degree of substitution of RAMEB is 12.6 methyl groups / cyclodextrin.
SBECD sulphobutyl ether β-cyclodextrin
IU international units. 1000 IU=25 μg of vitamin D.
TRIMEB heptakis(2,3,6-tri-O-methyl)-μ-cyclodextrin. The degree of substitution of TRIMEB is 21 methyl groups/cyclodextrin.

The Examples hereinbelow illustrate the invention.

### EXAMPLE 1

### Complex of Vitamin D₃ and RAMEB

### Example 1A

25 μg of cholecalciferol are mixed into 0.975 mg of RAMEB in water or tert-butanol; the solvent is then removed by spraying or lyophilisation.

### Example 1B

25 μg of cholecalciferol are mixed into 9.975 mg of RAMEB in water or tert-butanol; the solvent is then removed by spraying or lyophilisation.

### Example 1C

25 μg of cholecalciferol are mixed into 19.975 mg de RAMEB in water or tert-butanol; the solvent is then removed by spraying or lyophilisation.

### EXAMPLE 2

### Pharmaceutical Composition for a Sachet Containing 2 g of Strontium Ranelate and 1000 IU of Vitamin D₃

### Example 2A

The complex of vitamin D₃ and RAMEB of Example 1 A is mixed into 4 g of Protelos® granules containing 2 g of anhydrous strontium ranelate.

| | | |
|---|---|---|
| Anhydrous strontium ranelate | 2 | g |
| Cholecalciferol | 25 | μg |
| RAMEB | 0.975 | mg |
| Aspartame | 20 | mg |
| Maltodextrin | 400 | mg |
| Mannitol | 948 | mg |

### Example 2B

The complex of vitamin D₃ and RAMEB of Example 1B is mixed into 4 g of Protelos® granules containing 2 g of anhydrous strontium ranelate.

| | | |
|---|---|---|
| Anhydrous strontium ranelate | 2 | g |
| Cholecalciferol | 25 | μg |
| RAMEB | 9.975 | mg |
| Aspartame | 20 | mg |
| Maltodextrin | 400 | mg |
| Mannitol | 948 | mg |

### Example 2C

The complex of vitamin D₃ and RAMEB of Example 1 C is mixed into 4 g of Protelos® granules containing 2 g of anhydrous strontium ranelate.

| | | |
|---|---|---|
| Anhydrous strontium ranelate | 2 | g |
| Cholecalciferol | 25 | μg |
| RAMEB | 19.975 | mg |
| Aspartame | 20 | mg |
| Maltodextrin | 400 | mg |
| Mannitol | 948 | mg |

### EXAMPLE 3

### Tablet Containing 600 mg of Strontium Malonate and 500 IU of Vitamin D₃

### Example 3A

| | | |
|---|---|---|
| Anhydrous strontium malonate | 600 | mg |
| Cholecalciferol | 12.5 | μg |
| RAMEB | 487.5 | μg |
| Microcrystalline cellulose | 87 | mg |
| Polyvidone | 24 | mg |
| Anhydrous colloidal silica | 5 | mg |
| Magnesium stearate | 5 | mg |

### Example 3B

| | | |
|---|---|---|
| Anhydrous strontium malonate | 600 | mg |
| Cholecalciferol | 12.5 | μg |
| RAMEB | 9.9875 | mg |
| Microcrystalline cellulose | 87 | mg |
| Polyvidone | 24 | mg |
| Anhydrous colloidal silica | 5 | mg |
| Magnesium stearate | 5 | mg |

Preparation of the tablet of Example 3.
For about 5000 tablets:

3000 g of strontium malonate and 170 g of microcrystalline cellulose are carefully mixed. The mixture is screened, and then 120 g of polyvidone and purified water (q.s.p. to obtain a homogeneous granulate—about 375 g) are added. The granulate is screened, dried at 40° C. for 2½ to 3 hours, and then screened again.

25 g of anhydrous colloidal silica and 265 g of microcrystalline cellulose are carefully mixed and screened and then added to the previously prepared granulate and the complex of Example 1 (2.5 g of complex 1A when it is desired to prepare tablets according to Example 3A; 50 g of complex 1C when it is desired to prepare tablets according to Example 3B).

300 g of the resulting mixture are added to 25 g of screened magnesium stearate and then, when a homogeneous mixture is obtained, the rest of the mixture is added.

The final mixture is compressed.

### EXAMPLE 4

### Tablet Containing 798 mg of Strontium Acetate and 500 IU of Vitamin D₃

### Example 4A

| | | |
|---|---|---|
| Anhydrous strontium acetate | 798 | mg |
| Cholecalciferol | 12.5 | μg |
| RAMEB | 487.5 | μg |
| Microcrystalline cellulose | 116 | mg |
| Polyvidone | 32 | mg |
| Anhydrous colloidal silica | 6.66 | mg |
| Magnesium stearate | 6.66 | mg |

### Example 4B

| | | |
|---|---|---|
| Anhydrous strontium acetate | 798 | mg |
| Cholecalciferol | 12.5 | μg |
| RAMEB | 9.9875 | mg |
| Microcrystalline cellulose | 116 | mg |
| Polyvidone | 32 | mg |
| Anhydrous colloidal silica | 6.66 | mg |
| Magnesium stearate | 6.66 | mg |

Preparation of the tablet of Example 4.
For about 5000 tablets:

3990 g of strontium acetate and 227 g of microcrystalline cellulose are carefully mixed. The mixture is screened, and then 160 g of polyvidone and purified water (q.s.p. to obtain a homogeneous granulate—about 500 g) are added. The granulate is screened, dried at 40° C. for 2½ to 3 hours, and then screened again.

33.3 g of anhydrous colloidal silica and 353 g of microcrystalline cellulose are carefully mixed and screened and then added to the previously prepared granulate and the complex of Example 1 (2.5 g of complex 1A when it is desired to prepare tablets according to Example 4A; 50 g of complex 1C when it is desired to prepare tablets according to Example 4B).

400 g of the resulting mixture are added to 33.3 g of screened magnesium stearate and then, when a homogeneous mixture is obtained, the rest of the mixture is added.

The final mixture is compressed.

### EXAMPLE 5

### Tablet Containing 790 mg of Strontium Succinate and 500 IU of Vitamin D₃

### Example 5A

| | | |
|---|---|---|
| Anhydrous strontium succinate | 790 | mg |
| Cholecalciferol | 12.5 | μg |
| RAMEB | 487.5 | μg |
| Microcrystalline cellulose | 114.5 | mg |
| Polyvidone | 31.6 | mg |
| Anhydrous colloidal silica | 6.6 | mg |
| Magnesium stearate | 6.6 | mg |

### Example 5B

| | | |
|---|---|---|
| Anhydrous strontium succinate | 790 | mg |
| Cholecalciferol | 12.5 | μg |
| RAMEB | 9.9875 | mg |
| Microcrystalline cellulose | 114.5 | mg |
| Polyvidone | 31.6 | mg |
| Anhydrous colloidal silica | 6.6 | mg |
| Magnesium stearate | 6.6 | mg |

Preparation of the tablet of Example 5.
For about 5000 tablets:

3950 g of strontium succinate and 224 g of microcrystalline cellulose are carefully mixed. The mixture is screened, and then 158 g of polyvidone and purified water (q.s.p. to obtain a homogeneous granulate—about 500 g) are added. The granulate is screened, dried at 40° C. for 2½ to 3 hours, and then screened again.

33 g of anhydrous colloidal silica and 348 g of microcrystalline cellulose are carefully mixed and screened and then added to the previously prepared granulate and the complex of Example 1 (2.5 g of complex 1A when it is desired to prepare tablets according to Example 5A; 50 g of complex 1C when it is desired to prepare tablets according to Example 5B).

400 g of the resulting mixture are added to 33 g of screened magnesium stearate and then, when a homogeneous mixture is obtained, the rest of the mixture is added.

The final mixture is compressed.

### EXAMPLE 6

### Tablet Containing 900 mg of Strontium Ketoglutarate and 500 IU of Vitamin D₃

### Example 6A

| | | |
|---|---|---|
| Anhydrous strontium ketoglutarate | 900 | mg |
| Cholecalciferol | 12.5 | μg |
| RAMEB | 487.5 | μg |
| Microcrystalline cellulose | 130.5 | mg |
| Polyvidone | 36 | mg |
| Anhydrous colloidal silica | 7.5 | mg |
| Magnesium stearate | 7.5 | mg |

### Example 6B

| | | |
|---|---|---|
| Anhydrous strontium ketoglutarate | 900 | mg |
| Cholecalciferol | 12.5 | μg |
| RAMEB | 9.9875 | mg |
| Microcrystalline cellulose | 130.5 | mg |
| Polyvidone | 36 | mg |
| Anhydrous colloidal silica | 7.5 | mg |
| Magnesium stearate | 7.5 | mg |

Preparation of the tablet of Example 6.
For about 5000 tablets:

4500 g of strontium ketoglutarate and 255 g of microcrystalline cellulose are carefully mixed. The mixture is screened, and then 180 g of polyvidone and purified water (q.s.p. to obtain a homogeneous granulate—about 560 g) are added. The granulate is screened, dried at 40° C. for 2½ to 3 hours, and then screened again.

37.5 g of anhydrous colloidal silica and 397 g of microcrystalline cellulose are carefully mixed and screened and then added to the previously prepared granulate and the complex of Example 1 (2.5 g of complex 1 A when it is desired to prepare tablets according to Example 6A; 50 g of complex 1C when it is desired to prepare tablets according to Example 6B).

525 g of the resulting mixture are added to 37.5 g of screened magnesium stearate and then, when a homogeneous mixture is obtained, the rest of the mixture is added.

The final mixture is compressed.

### EXAMPLE 7

### Stability of the Vitamin D₃+RAMEB Complex of Example 1B

The stability of the vitamin D₃+RAMEB complex of Example 1B at 40° C./75% RH was tested and compare to the stability of: 1) pure vitamin D₃, 2) a concentrate of vitamin D₃ in powder form (DSM).

The study is carried out in pill containers of sealed amber glass (stopper of type for antibiotics, chlorobutyl, grey, D13—natural aluminium crimp-on cap D20 mm, tear-off lid).

| | % vitamin D₃ | | |
|---|---|---|---|
| | pure | vitamin D₃ | vitamin D₃ + RAMEB |
| t | vitamin D₃ | concentrate | complex (Example 1B) |
| t0 | 98.0 | 92.7 | 94.3 |
| t0 + 3 weeks | 20.3 | 87.4 | 93.6 |
| 40° C./75% RH | | | |
| t0 + 6 weeks | 23.1 | 88.1 | 94.3 |
| 40° C./75% RH | | | |

The table above shows that the stability of the complex of vitamin D₃ and RAMEB according to the invention is improved.

### EXAMPLE 8

### Stability of the Pharmaceutical Composition of Example 2B.

The stability of the pharmaceutical composition of Example 2B according to the invention in a sachet was tested under various temperature and humidity conditions.

The sachets are composed of a multilayer complex (paper/polyethylene/aluminium/polyethylene).

Sachet

| | vitamin D₃ content (IU) | | |
|---|---|---|---|
| t | 25° C./60% RH | 30° C./65% RH | 40° C./75% RH |
| t0 | 1011.8 | | |
| t0 + 6 weeks | 997.6 | 999.3 | 1014.9 |
| t0 + 3 months | 983.6 | 1000.0 | 986.1 |
| t0 + 6 months | 1017.9 | 998.2 | 998.0 |

The table above shows that the vitamin D contained in the sachet formulation of strontium ranelate, vitamin D and cyclodextrin according to the invention has excellent stability, even under conditions of high temperature and humidity (40° C./75% RH).

### EXAMPLE 9

### Uniformity of Content (of Vitamin D₃) of the Pharmaceutical Composition of Example 2B.

The test is carried out on 10 sachets.

The contents of each sachet are introduced into a conical flask, and then 25 ml of methanol are added. The mixture is stirred for 1 hour and then centrifuged for 10 mins. at 4000 revolutions per minute.

A reference solution of vitamin D₃ in methanol (concentration 1 μg/ml) is also prepared. The solutions under test are assayed using the technique of reverse-phase liquid chromatography with detection by UV spectrophotometry.

The vitamin D₃ content Xᵢ, of the i^{th} sachet (i being from 1 to 10) is calculated as follows:

Xᵢ=ATᵢ/AR

where ATᵢ, is the area under the vitamin D₃ peak for the i^{th} sachet, and AR is the area under the vitamin D₃ peak in the chromatogram of the reference solution.

The average content Xₘ is expressed as follows:

Xₘ=(ΣXᵢ)/10

The acceptance value (AV), expressed as a percentage of the theoretical value, is given by the following formula:

AV=(M−Xₘ)+kxs

where:
Xₘ is the average content, expressed as a percentage of the theoretical value;
M is the reference value, expressed as a percentage of the theoretical value: M=98.5 if Xₘ<98.5; M=Xₘ if 98.5 ≦Xₘ≦101.5; M=101.5 if Xₘ>101.5;
k is the acceptability constant (k=2.4 for 10 sachets);
s is the standard deviation of the content values Xᵢ.

### Results:

| Content uniformity parameters | Batch L0027602 (sachet) |
|---|---|
| (vitamin D₃) | according to Example 2B |
| Average content | 94.4% |
| Coefficient of variation | 2.3% |
| Acceptance value (AV) | 9.4 |

According to the European Pharmacopoeia, Article 2.9.40, an acceptance value of less than 15 means that the uniformity of content satisfies the requirements (level L1).

The table above therefore shows that the vitamin D contained in the sachet formulation of strontium ranelate, vitamin D and cyclodextrin according to the invention has a uniformity of content which meets regulatory requirements.

1. A pharmaceutical composition comprising, a strontium salt, vitamin D, a cyclodextrin, and one or more inert, non-toxic, pharmaceutically acceptable excipients or carriers.

2. The pharmaceutical composition of claim 1, wherein the vitamin D is cholecalciferol (vitamin D₃).

3. The pharmaceutical composition of claim 2, wherein the vitamin D₃ dose is 1000 IU.

4. The pharmaceutical composition of claim 1, wherein the cyclodextrin is a substituted β-cyclodextrin.

5. The pharmaceutical composition of claim 4, wherein the β-cyclodextrin is substituted by one or more methyl, hydroxypropyl or sulphobutyl ether groups.

6. The pharmaceutical composition of claim 5, wherein the substituted β-cyclodextrin is selected from hydroxypropyl-β-cyclodextrins (HPBCDs), sulphobutyl ether β-cyclodextrins (SBECDs) and methylated or partially methylated β-cyclodextrins.

7. The pharmaceutical composition of claim 6, wherein the substituted β-cyclodextrin is randomly methylated β-cyclodextrin (RAMEB).

8. The pharmaceutical composition of claim 1, wherein the weight ratio between the amount of vitamin D and the amount of cyclodextrin is between 1/40 and 1/800 inclusive.

9. The pharmaceutical composition of claim 1, wherein the strontium salt is selected from strontium ranelate, strontium malonate, strontium acetate, strontium L-ascorbate, strontium aspartate, strontium borate, strontium camphorate, strontium carbonate, strontium ketoglutarate, strontium citrate, strontium ethanesulphonate, strontium formate, strontium fumarate, strontium gluconate, strontium glutamate, strontium hydrogen phosphate, strontium lactate, strontium L-lactate, strontium L-malate, strontium maleate, strontium methanesulphonate, strontium nitrate, strontium oxalate, strontium phosphate, strontium propanesulphonate, strontium succinate, strontium sulphate, strontium tartrate and hydrates thereof

10. The pharmaceutical composition of claim 9, wherein the strontium salt is strontium ranelate.

11. The pharmaceutical composition of claim 1 which is in the form of a swallowable tablet, chewable tablet, effervescent tablet, dispersible tablet or granules.

12. The pharmaceutical composition of claim 11 in the form of granules for a sachet.
